(19) United States
(12) Patent Application Publication
(10) Pub. No.: US 2020/0223772 A1
(43) Pub. Date: Jul. 16, 2020

(54) NOVEL METHOD FOR FLUORINATING CHLOROALKANES

(71) Applicant: THE CHEMOURS COMPANY FC, LLC

(72) Inventor: Xuehui SUN, Wilmington, DE (US)

(73) Assignee: THE CHEMOURS COMPANY FC, LLC, Wilmington, DE (US)

(21) Appl. No.: 15/759,035

(22) PCT Filed: Sep. 9, 2016
(86) PCT No.: PCT/US2016/050912

§ 371 Date: Mar. 9, 2018

(51) Int. Cl. C07C 17/20

(52) U.S. Cl. CPC C07C 17/202

(57) ABSTRACT

The disclosure relates to a method for fluorinating an alkane substrate comprising contacting said alkane substrate with a fluoroalkane in the presence of a fluorination catalyst selected from chrome oxide, fluorinated chrome oxide, aluminum oxide and fluorinated aluminum oxide at elevated temperatures.

### BACKGROUND OF THE DISCLOSURE

This disclosure relates to novel methods for preparing fluorinated organic compounds, and more particularly to methods of producing fluorinated hydrocarbons.

Hydrofluorocarbons (HFCs), in particular hydrofluoroalkenes, such as tetrafluoropropenes (including 2,3,3,3-tetrafluoropropene (HFO-1234yf or 1234yf)) have been disclosed to be effective refrigerants, fire extinguishants, heat transfer media, propellants, foaming agents, blowing agents, gaseous dielectrics, sterilant carriers, polymerization media, particulate removal fluids, carrier fluids, buffing abrasive agents, displacement drying agents and power cycle working fluids. Unlike chlorofluorocarbons (CFCs) and hydrochlorofluorocarbons (HCFCs), both of which potentially damage the Earth's ozone layer, HFCs do not contain chlorine and, thus, pose no threat to the ozone layer.

In addition to ozone depleting concerns, global warming is another environmental concern in many of these applications. Thus, there is a need for compositions that meet both low ozone depletion standards as well as having low global warming potentials. Certain fluoroolefins are believed to meet both goals. Thus, there is a need for manufacturing processes that provide halogenated hydrocarbons and fluoroolefins that contain no chlorine that also have a low global warming potential.

One such tetrafluoropropene that has no chlorine and has a low global warming potential is 2,3,3,3-tetrafluoro-1-propene (HFO-1234yf or 1234yf). The preparation of HFO-1234yf generally includes at least three reaction steps, as follows:
(i) (CQ₂=CCl—CH₂Q or CQ₃-CCl═CH₂ or CQ₃-CHCl—CH₂Q)+HF→2-chloro-3,3,3-trifluoropropene (HCFO-1233xf or 1233xf)+HCl in a vapor phase reactor charged with a solid catalyst;
(ii) 2-chloro-3,3,3-trifluoropropene (HCFO-1233xf)+HF→2-chloro-1,1,1,2-tetrafluoropropane (HCFC-244bb or 244bb) in a liquid phase reactor charged with a liquid hydrofluorination catalyst; and
(iii) 2-chloro-1,1,1,2-tetrafluoropropane (HCFC-244bb)→2,3,3,3-tetrafluoropropene (HFO-1234yf) in a vapor phase reactor.
   wherein Q is independently selected from F, Cl, Br, and I, provided that at least one X is not fluorine.

However, the direct fluorination of 2-chloro-3,3,3-trifluoropropene (HCFO-1233xf) by HF requires not only high temperatures, but also results in excessive fluorination thereof, thereby forming 1,1,1,2,2-pentafluoropropane (HFC-245cb or 245cb). The formation of 245cb decreases the yield of 1234yf. To avoid this problem using HF, the hydrofluorination of 1233xf is conducted under mild temperatures using antimony pentachloride or a fluorinated antimony pentachloride as the catalyst. Unfortunately, the process of converting 1233xf to 1,1,1,2-tetrafluoro-2-chloropropane (HCFC-244bb or 244bb) at mild temperature in the presence of SbCl₅, is difficult and expensive to perform.

### SUMMARY OF THE DISCLOSURE

The present process is directed to a new method for fluorinating an alkane and for making refrigerants, such as 1234yf and 1,3,3,3-tetrafluoropropene (HFO-1234ze or 1234ze) and useful intermediates thereof. More specifically, a process has been developed to use a fluorocarbon instead of HF to fluorinate alkane substrates, including fluorochlorocarbons.

The disclosure relates to a method for fluorinating an alkane substrate with a fluoroalkane in the presence of a fluorination catalyst at an elevated temperature in the absence of hydrogen fluoride. In an embodiment, this process is useful for fluorinating 1,1,1-trifluoro-2,3-dichloropropane (243db) to form 1,1,1,2-tetrafluoro-3-chloropropane (244eb), which, in turn is dehydrochlorinated to form 1234yf.

### DETAILED DESCRIPTION OF THE DISCLOSURE

The foregoing general description and the following detailed description are exemplary and explanatory only and are not restrictive of the invention.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

Unless otherwise defined, all technical and scientific terms used herein have the same meaning as commonly understood by one of ordinary skill in the art to which this invention belongs. In case of conflict, the present specification, including definitions, will control. Although methods and materials similar or equivalent to those described herein can be used in the practice or testing of embodiments of the present invention, suitable methods and materials are described below. In addition, the materials, methods, and examples are illustrative only and not intended to be limiting.

Unless indicated to the contrary, the term “alkane” refers to a saturated compound comprised of carbon and hydrogen atoms and containing 1-8 carbon atoms which may be unsubstituted or substituted with fluorine and optionally other halogens or a leaving group as defined herein.

The term “alkane substrate” refers to an alkane having 1-8 carbon atoms having at least one leaving group thereon, such as halogen, Cl, Br, or I, tosylates, mesylates brosylate, nosylate, mesylate, trifluoromethanesulfonate, nonafluorobutanesulfonate, 2,2,2-trifluoroethanesulfonate, and the like. In an embodiment, the leaving group on the alkane substrate is a chlorine atom.

The term “perfluorinated alkyl group”, as used herein, means an alkyl group wherein all hydrogens on carbon atoms have been substituted by fluorines. Examples of a perfluorinated alkyl group include —CF₃ and —CF₂CF₃.

As used herein, the term “fluoroalkane” denotes a compound containing carbon, fluorine, hydrogen and optionally chlorine.

The term “fluorochlorocarbon” denotes a compound containing carbon, hydrogen, chlorine and fluorine.

The term “fluoroolefin”, as used herein, denotes a compound containing hydrogen, carbon, fluorine, and at least one carbon-carbon double bond and optionally chlorine.

The term “dehydrohalogenation” (or dehydrohalogenating), as used herein, refers to the removal of hydrogen chloride (HCl) or hydrogen fluoride (HF) from a chlorofluorocarbon or a hydrofluorocarbon.

As used herein, the term “conversion” with respect to a reactant, which typically is a limiting agent, refers to the number of moles reacted in the reaction process divided by the number of moles of that reactant initially present in the process.

Described is a method for fluorinating an alkane substrate using a fluoroalkane in the presence of a fluorination catalyst. The fluoroalkane contains carbon, hydrogen and fluorine atoms. The fluorine atoms may be part of a perfluorinated alkyl group. However, in addition, the fluoroalkane may contain at least one additional fluorine atom substituted on a carbon atom on another part of the molecule. In an embodiment, the at least one fluorine atom is substituted on an internal carbon atom, i.e. not on the terminal carbon. In another embodiment, the at least one fluorine atom is substituted on a terminal carbon atom. In another embodiment, fluorine atoms are substituted on both internal carbon atoms and terminal carbon atoms. In addition to the fluorine atoms on the perfluorinated alkyl portion of the molecule, the fluoroalkane may contain 1, 2, 3, 4, 5, 6 or more additional fluorine atoms, depending on the total number of carbon atoms.

In an embodiment, the fluoroalkane has the formula: (R1)CF(R3)(R2), where R1 and R2 are independently perfluorinated alkyl having 1 to 8 carbon atoms or hydrogen, and R3 is hydrogen or fluorine. In an embodiment, R1 and R2 have 1-3 carbon atoms.

In an embodiment, the fluoroalkane has the formula R1CF₂H, where R1 is as defined hereinabove. In another embodiment, the fluoroalkane has the formula: R1CH₂F, wherein R1 is as defined hereinabove. In still another embodiment, the fluoroalkane has the formula R1CF₂R2, wherein R1 and R2 are as defined hereinabove. In a further embodiment, the fluoroalkane has the formula R1CHFR2, wherein R1 and R2 are as defined hereinabove. In one embodiment, the fluoroalkane has the formula R1CHFCH₂R2, or R1CHFCHFR2, or R1CHFCF₂R2, or R1CF2CH2R2, or R1CF₂CHFR2, or R1CF₂CF₂R2, or R1CH₂CHFR2 or R1CH₂CF₂R2, wherein R1 and R2 are as defined hereinabove. Examples include 1,1,1,3,3-pentafluoropropane, 1,1,1,2,3-pentafluoropropane, difluoromethane, and the like.

The fluoroalkane is either a known compound or prepared by techniques known in the art.

The alkane substrate is an alkane having 1-8 carbon atoms. It has at least one leaving group, as defined above, substituted on one of the carbon atoms. In an embodiment, the leaving group is a chlorine atom. It may also have more than one fluorine atom thereon, including perfluorinated groups. Examples include 1,1,1-trifluoro-2,3-dichloropropane; 1,1-difluoro-1,2,3-trichloropropane; 1,1,1-trifluoro-3,3-dichloropropane; 1,1,1-trifluoro-2,2-dichloropropane and the like.

The fluorination reaction described herein may be conducted in any reactor suitable for a vapor phase fluorination reaction. The reactor is made of a material that is resistant to reactants employed. The reactor may be constructed from materials which are resistant to the corrosive effects of hydrogen fluoride such as stainless steel, Hastelloy, Inconel, Monel, gold or gold-lined or quartz. The reaction described herein may be conducted batchwise, continuous, or semi-continuous or combination thereof. Suitable reactors include batch reactor vessels and tubular reactors.

The fluorination reaction in the present process is conducted in the vapor phase. The reactor is filled with a vapor phase fluorination catalyst. Any fluorination catalysts used in the vapor phase known in the art may be used in this process. Suitable catalysts include, but are not limited to chromium, aluminum, cobalt, manganese, nickel and iron oxides, hydroxides, halides, oxyhalides, inorganic salts thereof and mixtures thereof, any of which may be optionally halogenated. In an embodiment, the catalyst is a chrome catalyst, i.e., a catalyst comprised of chromium. A chrome catalyst can be a chrome halide, such as fluoride, chloride or bromide, or a chromium oxide, such as Cr₂O₃, which may be unsupported or supported on carbon or aluminum oxide. The term “chrome catalyst” includes chromium (III) catalyst, where chromium is in the +3 oxidation step. For example, the catalyst may be a chromium (III) halide, such as CrCl₃, CrBr₃, CrF₃, and the like or a chromium oxide catalyst e.g., Cr₂O₃, capable of catalyzing a fluorination reaction. The chromium catalyst may be mixed with other metals, such as zinc, for example, Cr₂O₃ mixed with zinc, e.g., containing from about 1% to about 10% (w/w) zinc, such as about 5% zinc mixed with Cr₂O₃ (w/w). The chrome oxide catalyst may comprise, for example, a chromium oxide catalyst or a chromium oxyfluoride represented by the formula Cr₂OₓF_{y}, where x+y/2=3 and x is an integer 0, 1, 2 or 3 and y is an integer of 0, 1, 2, 3, 4, 5 or 6. The chromium may also be present in oxidation states other than chromium (III), such as 2, 4, 5 or 6, e.g., ClO₂.

Combinations of catalysts suitable for the present disclosure nonexclusively include, FeCl₃/C, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃, CrCl₃/carbon, CrCl₃/Al₂O₃ and mixtures thereof. Chromium oxide/aluminum oxide catalysts are described in U.S. Pat. No. 5,155,082, the contents of which are incorporated herein by reference. In an embodiment, chromium (III) oxides such as crystalline chromium oxide or amorphous chromium oxide is the catalyst used in the fluorination reaction described herein. Chromium oxide (Cr₂O₃) is a commercially available material which may be purchased in a variety of particle sizes. The fluorination catalyst is present in at least an amount sufficient to catalyze the reaction.

In an embodiment, the catalyst is a chrome catalyst, a fluorinated chrome catalyst, aluminum oxide catalyst or fluorinated aluminum oxide. In some embodiments of the present disclosure, the catalyst is selected from chromium oxide, fluorinated chromium oxide, aluminum oxide, fluorinated aluminum oxide, chrome halide, which catalyst may be unsupported or supported, for example, on carbon, aluminum fluoride supports, and when the catalyst is other than aluminum oxide, aluminum oxide can be used as the support. Examples of catalyst that could be used are Cr₂O₃, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃, CrCl₃, CrCl₃/carbon and mixtures thereof. The catalyst can be used with or without additional elements, such as alkali metal, alkaline earth metal, and transition metals, such as zinc and the like.

The reaction is effected at a time sufficient for the alkane substrate to be in contact with the fluoroalkane in the presence of the fluorination catalyst. A measure of this reaction time is the contact time. As used herein, the contact time (CT) is defined as volume of catalyst/reactor flow rate of gas components flowed through the reaction system.

In the present specification, the contact time of the reaction according to the present invention is defined by referring to the volume of the loading (catalyst) which is represented by A and the volume of the raw material gas introduced into the reactor per second is represented by B. The value of B is calculated from the number of moles of the raw material introduced per second, pressure and temperature. The value (=A/B) is determined by dividing A by B and this is defined as “contact time”. In the reactor, gases other than the target product are produced as by-products to cause change in the number of moles, but these are not considered upon calculating “contact time”.

Contact time depends on the temperature (reaction temperature) and the pressure of operation and the volume of the loading (catalyst). Therefore, it is desirable to suitably adjust the supply rate (contact time) of the reaction raw material to determine the optimum value for each of the predetermined temperature, pressure, and the volume of the loading (catalyst).

In the reaction of the present invention, the contact time is ranges from about 1 second to about 20 min. In one embodiment, this contact time ranges from about 2 seconds to about 15 min. In another embodiment, contact time ranges from about 5 second to about 10 min.

The catalyst, in one embodiment, is activated with HF and nitrogen. However, before conducting the reaction, any free HF is removed.

The fluorination reaction is conducted in the absence of hydrogen fluoride. The fluorinating agent in the present process is the fluoroalkane. No other fluorinating agent is necessary.

The fluorination reaction may be conducted in the presence or absence of oxygen. In an embodiment, it is conducted in the presence of oxygen. In an embodiment, it is conducted in the absence of oxygen and in the presence of an inert gas, such as nitrogen, argon or helium or a combination thereof.

The fluoroalkane and the alkane substrate are present in effective amounts for the fluorination to occur. The molar ratio of alkane substrate to fluoroalkane ranges from about 0.1 to about 10, and in another embodiment from about 0.5 to about 5. In one embodiment, the reaction takes place at a temperature of about 150° C. to about 400° C. In another embodiment, the reaction takes place at a temperature in the range of from about 200° C. to about 350° C. In one embodiment, the hydrofluorination described hereinabove is conducted in a reaction vessel at about 150° C., about 180° C., about 200° C., about 225° C., about 240° C., about 250° C., about 275° C., about 280° C., about 300° C., about 320° C., or about 350° C.

The reaction is conducted at effective pressures. In one embodiment, the pressure ranges from about 0 to about 120 psig, and in another embodiment, it ranges from about 10 to about 100 psig and in a third embodiment, it ranges from about 20 to about 80 psig.

In the fluorination reaction, a fluorine atom on the fluoroalkane substitutes for the leaving group substituent on the alkane substrate, thereby fluorinating the alkane substrate. In an embodiment, if the alkane substrate has more than one leaving group, such as chlorine atoms, the fluoroalkane may substitute one or more fluoro atoms for one or some or all of the leaving groups, such as chlorine atoms on the alkane substrate, as illustrated below.

The fluorinated alkane substrate is separated from the reaction mixture by techniques known in the art, such as by distillation and the like.

The advantage of this process is that intermediates in the process for forming refrigerants, such as 1234yf, may be formed without using large quantities of HF. Thus, this process reduces the need for HF and the need to recycle HF. In addition, since the present process is conducted in the absence of HF, the corrosion associated with HF is at least minimized or completely avoided. Moreover, this process avoids the need for scrubbing, which is usually accompanied by the use of HF for hydrofluorination.

For example, in an embodiment, the first step in the formation of 1234yf is the monofluorination of 243db with a fluoroalkane and the fluorination catalyst, in accordance with the process described herein. The product, 244eb, is separated from the reaction mixture.

The 244eb is then dehydrochlorinated in the vapor phase in a second reactor with base with or without a dehydrochlorination catalyst to form 1234yf. In an embodiment, 244eb is fed to a second vapor phase reactor (dehydrochlorination reactor) to be dehydrochlorinated to make the desired product 2,3,3,3-tetrafluoropropene (HFO-1234yf). This reactor contains either no catalyst or a catalyst that can catalytically dehydrochlorinate HCFC-244eb to make HFO-1234yf.

If a catalyst is present in the dehydrochlorination reaction, the catalysts may be metal halides, halogenated metal oxides, neutral (or zero oxidation state) metal or metal alloy, or activated carbon in bulk or supported form. Metal halide or metal oxide catalysts may include, but are not limited to, mono-, bi-, and tri-valent metal halides, oxides and their mixtures/combinations, and more preferably mono-, and bi-valent metal halides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Component halides include, but are not limited to, F⁻, Cl⁻, Br⁻, and I⁻. Examples of useful mono- or bi-valent metal halide include, but are not limited to, LiF, NaF, KF, CsF, MgF₂, CaF₂, LiCl, NaCl, KCl, and CsCl. Halogenation treatments can include any of those known in the prior art, particularly those that employ HF, F₂, HCl, Cl₂, HBr, Br₂, HI, and I₂ as the halogenation source.

When neutral, i.e., zero valent, metals, metal alloys and their mixtures are used in the dehydrochlorination reaction. Useful metals include, but are not limited to, Pd, Pt, Rh, Fe, Co, Ni, Cu, Mo, Cr, Mn, and combinations of the foregoing as alloys or mixtures. The catalyst may be supported or unsupported. Useful examples of metal alloys include, but are not limited to, SS 316, Monel 400, Inconel 825, Inconel 600, Inconel 625, and the like.

In an embodiment, catalysts for the dehydrochlorination reaction include activated carbon, stainless steel (e.g., SS 316), austenitic nickel-based alloys (e.g., Inconel 625), nickel, fluorinated 10% CsCl/MgO, 10% CsCl/MgF₂ and the like. A suitable reaction temperature ranges from about 300 to about 550° C. and a suitable reaction pressure may range from about 0 to about 150 psig. The reactor effluent may be fed to a caustic scrubber or to a distillation column to remove the byproduct of HCl to produce an acid-free organic product which, optionally, may undergo further purification using one or any combination of purification techniques that are known in the art.

A method of producing 1234yf, in accordance with the process of the present disclosure is by converting 243db to 244eb using a fluorinated alkane, in accordance with the present invention, the products of which can easily be converted to 1234yf, by fluorinating using the present process, the use of the corrosive HF is avoided.

As another example, 1,3,3,3-tetrafluoropropene (HFO-1234ze) can be prepared by this method. In an embodiment, CCl₃CH₂CHClF (HCFC-241fb) is prepared by techniques known in the art, such as described in US Publication No. 2013/0211156, the contents of which are incorporated by reference. The HCFC-241fb is then fluorinated using a fluorinated alkane substrate, in accordance with the process described herein, to form CF₃CH₂CHClF, which can then be dehydrochlorinated in the presence of a dehydrochlorination catalyst to form HFO-1234ze.

In another embodiment, HFO-1234ze can be formed by reacting CF₃CH₂CHCl₂, which is prepared by techniques known in the art, with a fluorinated alkane substrate, in accordance with the process described herein, to afford CF₃CH₂CHClF, which can be dehydrochlorinated by techniques known in the art using a dehydrochlorination catalyst, such as activated carbon, to produce HFO-1234ze.

In another example, 1,1,1-trifluoro-2,3-dichloropropane is fluorinated with a fluorinated alkane substrate, in accordance with the present invention, to afford 1,1,1,3-tetrafluoro-2-chloropropane and 1,1,1,2-tetrafluoro-3-chloropropane. The two products can be separated and then separately dehydrochlorinated by techniques known in the art to produce 1,3,3,3-tetrafluopropene (1234ze) and 2,3,3,3-tetrafluoropropene (1234yf), respectively.

The following non-limiting examples further illustrate the invention. In the examples, 243db is 1,1,1-trifluoro-2,3-dichloropropane; 244bb is 1,1,1,2-tetrafluoro-2-chloropropane; 244db is 1,1,1,3-tetrafluoro-2-chloropropane; 244eb is 1,1,1,2-tetrafluoro-3-chloropropane; 245eb is 1,1,1,2,3-pentafluoropropane; 245fa is 1,1,1,3,3-pentafluoropropane; 1233xf is 3,3,3-trifluoro-2-chloropropene; 1233zd is 3,3,3-trifluoro-1-chloropropene; 1234yf is 2,3,3,3-tetrafluoropropene; 1234ze is 1,3,3,3-tetrafluoropropene;

### EXAMPLES

### Example 1—Fluorination of 243db by 245fa to make 244eb

A half-inch Hastelloy tube was loaded with 8 mL JM 62-2 chrome catalyst (Cr₂O₃) purchased from Johnson Matthey, and was doped with 4,500 ppm Na. The catalyst was activated with HF and N₂. A mixture of 243db and 245fa was then fed into the reactor at a rate of 1 mL/hr and temperatures of 225° C., 250° C. and 275° C. at 20 psig. The reactor effluent was analyzed by GC-MS. The composition of the feed material can be found in Table 1. The GC-MS data of the products obtained from the fluorination reaction can be found in Table 2.

TABLE 1

| compounds | GC area % |
|---|---|
| 245fa | 46.50% |
| 243db | 52.96% |
| others | 0.53% |

TABLE 2

| | GC area % | | |
|---|---|---|---|
| Compounds | 225° C. | 250° C. | 275° C. |
| 1234yf | 0.000% | 0.123% | 0.828% |
| 1234ze | 1.266% | 2.645% | 5.625% |
| 245fa | 48.696% | 45.436% | 36.566% |
| 1234ze | 3.080% | 0.873% | 1.117% |
| 1233xf | 3.381% | 10.191% | 23.427% |
| 1233zd | 0.577% | 9.514% | 21.414% |
| 244eb | 0.694% | 1.879% | 2.181% |
| 1233zd | 0.369% | 1.118% | 2.884% |
| 243db | 41.937% | 27.276% | 5.958% |
| others | 0.000% | 0.944% | 0.000% |

### Example 2—Fluorination of 243db by 245eb to make 244eb and 244db

A half-inch Hastelloy tube was loaded with 8 mL JM 62-3 chrome catalyst (Cr₂O₃ mixed with 5% zinc by weight) purchased from Johnson Matthey. The catalyst was activated with HF and N₂. Then a mixture of 243db and 245eb was fed into the reactor at a rate of 1 mL/hr and temperatures of 200° C., 240° C., 280° C. and 320° C. at atmospheric pressure. The effluent was analyzed by GC-MS. The composition of the feed material can be found in Table 3, and the GC-MS data of the products can be found in Table 4.

TABLE 3

| Compounds | GC area % |
|---|---|
| 245eb | 53.25% |
| 243db | 42.98% |
| 244eb | 1.72% |
| 244db | 1.00% |
| others | 1.05% |

TABLE 4

| Temp | Mole Percents | | | | | | | | | | | |
|---|---|---|---|---|---|---|---|---|---|---|---|---|
| ° C. | 1234yf | 1234ze | 245fa | 245eb | 244bb | 1233xf | 1233zd-E | 244eb | 244db | 1233zd-Z | 243db | others |
| 200 | 6.19% | 0.00% | 0.72% | 14.32% | 0.00% | 27.45% | 0.60% | 33.23% | 4.59% | 0.00% | 12.67% | 0.22% |
| 200 | 6.29% | 0.00% | 0.75% | 14.17% | 0.00% | 28.06% | 0.60% | 33.22% | 4.46% | 0.00% | 12.23% | 0.22% |
| 200 | 6.40% | 0.00% | 0.76% | 14.04% | 0.00% | 28.49% | 0.61% | 33.25% | 4.34% | 0.00% | 11.89% | 0.22% |
| 240 | 14.45% | 0.52% | 1.90% | 1.58% | 0.24% | 54.39% | 2.71% | 18.54% | 0.60% | 0.30% | 4.60% | 0.17% |
| 240 | 14.31% | 0.53% | 1.88% | 1.71% | 0.23% | 53.45% | 2.63% | 19.42% | 0.64% | 0.29% | 4.74% | 0.17% |
| 240 | 14.01% | 0.51% | 1.79% | 1.99% | 0.21% | 51.72% | 2.47% | 21.07% | 0.73% | 0.27% | 5.05% | 0.18% |
| 280 | 18.66% | 0.95% | 0.95% | 0.29% | 0.18% | 70.97% | 5.81% | 0.95% | 0.00% | 0.74% | 0.52% | 0.00% |
| 280 | 18.84% | 0.95% | 0.95% | 0.30% | 0.17% | 70.63% | 5.83% | 1.02% | 0.00% | 0.75% | 0.56% | 0.00% |
| 280 | 18.82% | 0.96% | 0.96% | 0.31% | 0.17% | 70.53% | 5.85% | 1.08% | 0.00% | 0.74% | 0.58% | 0.00% |
| 320 | 19.15% | 1.14% | 0.34% | 0.34% | 0.00% | 71.16% | 6.64% | 0.00% | 0.00% | 0.96% | 0.00% | 0.27% |
| 320 | 19.21% | 1.16% | 0.34% | 0.33% | 0.00% | 71.04% | 6.69% | 0.00% | 0.00% | 0.96% | 0.00% | 0.26% |
| 320 | 19.30% | 1.18% | 0.35% | 0.35% | 0.00% | 70.86% | 6.73% | 0.00% | 0.00% | 0.97% | 0.00% | 0.25% |

### Comparative Example 1—Fluorination of 243db by HF to make 244eb and 244db

A half-inch Hastelloy tube was loaded with 8 mL JM 62-3 chrome catalyst. The catalyst was activated with HF and N₂. Then a mixture of 243db was fed into the reactor at a rate of 1 mL/hr with 10 sccm HF and temperatures of 200° C., 240° C. and 280° C. at atmospheric pressure. The effluent was analyzed by GC-MS. The GC-MS data of the products can be found in Table 5. The data shows that 243db was mainly converted to 1233xf.

TABLE 5

| Temp | Mole Percent | | | | | | | | | |
|---|---|---|---|---|---|---|---|---|---|---|
| ° C. | 1234yf | 244bb | 1233xf | 1233zd-E | 244eb | 244db | 1233zd-Z | 243db | 233ab | others |
| 200 | 0.00% | 0.18% | 44.65% | 0.55% | 0.37% | 0.00% | 0.00% | 51.52% | 0.49% | 2.24% |
| 240 | 0.20% | 0.10% | 66.24% | 1.09% | 0.29% | 0.00% | 0.12% | 27.38% | 0.73% | 3.85% |
| 280 | 0.25% | 0.00% | 74.54% | 1.73% | 0.29% | 0.00% | 0.22% | 16.57% | 0.86% | 5.54% |

Many aspects and embodiments have been described and are merely exemplary and not limiting. After reading the specification, skilled artisans appreciate that other aspects and embodiments are possible without departing from the scope of the invention.

Other features and benefits of any one or more of the embodiments will be apparent from the hereinabove detailed description and the claims.

What is claimed is:

1. A method for fluorinating an alkane substrate comprising contacting in the vapor phase said alkane substrate with a fluoroalkane in the presence of a fluorination catalyst at an elevated temperature to effect fluorination of said alkane substrate in the absence of HF or other fluorinating agent in the presence or absence of oxygen.

2. The method of claim 1 wherein the catalyst is selected from chrome oxide, fluorinated chrome oxide, aluminum oxide, fluorinated aluminum oxide and chrome halide.

3. The method of claim 2 wherein the catalyst is supported on carbon or on aluminum fluoride.

4. The method of claim 1 wherein the catalyst is chrome oxide or fluorinated chrome oxide.

5. The method of claim 1 wherein the catalyst is chrome halide and the catalyst is supported on aluminum oxide or carbon.

6. The method of claim 1, wherein said catalyst is Cr₂O₃, Cr₂O₃/Al₂O₃, Cr₂O₃/AlF₃, CrCl₃karbon, CoCl₂/Cr₂O₃/Al₂O₃, NiCl₂/Cr₂O₃/Al₂O₃, CoCl₂/AlF₃, NiCl₂/AlF₃, or mixtures thereof.

7. The method of claim 5 wherein one or more alkali metal, alkaline earth metal, transition metal or a combination thereof are additionally present.

8. The method of claim 1 wherein the catalyst is a chrome catalyst that is optionally doped with sodium.

9. The method of claim 1, wherein the fluorocarbon has the formula: (R1)CF(R3)(R2), where R1 and R2 are independently fluorinated alkyl having 1 to 8 carbon atoms or hydrogen, and R3 is hydrogen or fluorine.

10. The method of claim 8, wherein R1 and R2 have 1-3 carbon atoms.

11. The method of claim 1, wherein the fluorocarbon is R1CF₂H, or R1CH₂F, or R1CF₂R2, R1CHFR2, or R1CHFCH₂R2, or R1CHFCHFR2, or R1CHFCF₂R2, or R1CF2CH2R2, or R1CF₂CHFR2, or R1CF₂CF₂R2, or R1CH₂CHFR2 or R1CH₂CF₂R2.

12. The method of claim 1, wherein the molar ratio of alkane substrate to fluoroalkane ranges from about 0.1 to about 10.

13. The method of claim 1, wherein the molar ratio of alkane substrate to fluoroalkane ranges from about 0.5 to about 5.

14. The method of claim 1, wherein the temperature for the fluorination ranges from about 150° C. to about 400° C.

15. The method of claim 9, wherein the fluorination is effected at a temperature in the range of from about 200° C. to about 350° C.

16. The method of claim 1, wherein an inert gas is additionally present.

17. The method of claim 15, wherein the inert gas is nitrogen, helium or argon.

18. The method according to claim 1, wherein the fluorination is conducted at a pressure ranging from about 0 to about 100 psig.

19. The method according to claim 13, wherein the fluorination is conducted at a pressure ranging from about 10 to about 80 psig.

20. The method of claim 1, wherein the alkane substrate is 1,1,1-trifluoro-2,3-dichloropropane, 1,1,1-trifluoro-3,3-dichloropropane, 1,1,1-trifluoro-2,2-dichloropropane or 1,1-difluoro-1,2,3-trichloropropane.

21. The method of claim 1 having a contact time of said fluorination catalyst with said alkane substrate and said fluoroalkane ranges from about 1 second to about 20 min.

22. The method of claim 20 wherein the contact time ranges from about 2 seconds to about 15 minutes.

23. The method of claim 21 wherein the contact time ranges from about 5 seconds to about 10 minutes.

24. The method of claim 4 wherein the chrome oxide or fluorinated chrome oxide is mixed with zinc.

25. A method of preparing 2,3,3,3-tetrafluoropropene which comprises (a) fluorinating 1,1,1-trifluoro-2,3-dichloropropane with a fluorocarbon in accordance with the method of any one of claims 1-14 and isolating the 1,1,1,2-tetrafluoro-3-chloropropane produced therefrom and (b) dehydrochlorinating the 1,1,1,2-tetrafluoro-3-chloropropane to produce 2,3,3,3-tetrafluoropropene.
